(19) United States
(12) Patent Application Publication
(10) Pub. No.: US 2003/0023199 A1
(43) Pub. Date: Jan. 30, 2003

(54) P.A. link off system

(76) Inventor: Tim Heilizer, Thiensville, WI (US)

Correspondence Address: Tim Heilizer, 509 PARK CREST DR., THIENSVILLE, WI, 53092, US

(21) Appl. No.: 09/917,590

(22) Filed: Jul. 30, 2001

(51) Int. Cl. A61M 5/00

(52) U.S. Cl. 604/8

(57) ABSTRACT

Intraoperative bleeding from the inferior vena cava and/or the post hepatic vena cava that cannot be controlled can result in the patient's death. The ‘P.A. (peritoneal-atrial) Link Off System’ is a new surgical device that allows the surgeon to control bleeding from the inferior vena cava and/or the post hepatic vena cava. The ‘P.A. Link Off System’ is comprised of a new plastic tube ‘shunt’ and a new vascular clamp. A 26 cm clear plastic tube with three inflatable cuffs at different lengths along the tube. This is inserted into the inferior vena cava, below the renal veins. Once the cuffs are inflated, a vascular clamp, that allows closure of the inferior vena cava without closing the lumens of the 5 tubes, is placed. This allows control and isolation of the post hepatic vena cava.

### BACKGROUND

Intraoperative bleeding from the inferior vena cava and/or the post hepatic vena cava that cannot be controlled can result in the patient's death.

### BRIEF SUMMARY

The ‘P.A. (peritoneal-atrial) Link Off System’ is a surgical device that allows the surgeon to control bleeding from the inferior vena cava and/or the post hepatic vena cava.

### BRIEF DESCRIPTION

The ‘P.A. Link Off System’ is comprised of a plastic tube ‘shunt’ and a vascular clamp. A 26 cm clear plastic tube with three inflatable cuffs at different lengths along the tube. A vascular clamp that allows closure of the inferior vena cava without closing the lumens of the 5 tubes.

### DRAWINGS

See FIGS.: 1-6

FIG. 1: ‘P.A. Link Off’ shunt (2 views)

FIG. 2: ‘P.A. Link Off’ vascular clamp

FIG. 3: ‘P.A. Link Off’ shunt into inferior vena cava

FIG. 4: ‘P.A. Link Off’ shunt in inferior vena cava with cuffs inflated

FIG. 5: ‘P.A. Link Off’ shunt removal from inferior vena cava

FIG. 6: Inferior vena cava repair

### DETAILED DESCRIPTION

How it is Made:

‘P.A. Link Off ’ Shunt

A clear silastic tube. 26 cm in length, 1 cm in diameter and 1 mm thickness of silastic.

From:

0-1 cm. A 45-degree angled beveled blunted tip.

1-4 cm. A thin plastic inflatable rounded cuff around the outside of the silastic tube. When inflated with 15 cc of air, the cuff size is 3 cm by 3 cm by 3 cm.

12-14 cm. A thin plastic inflatable rounded cuff around the outside of the silastic tube. When inflated with 10 cc of air, the cuff size is 2 cm by 3 cm by 3 cm.

18-20 cm. A thin plastic inflatable rounded cuff around the outside of the silastic tube. When inflated with 10 cc of air, the cuff size is 2 cm by 3 cm by 3 cm.

21 cm. To inflate the cuff at 1-4 cm, there is a different smaller silastic tube attached to the outside of the longer (26 cm) tube. The smaller tube is 2 mm in diameter and is attached to the cuff at one end and a ball valve mechanism at the other end. The ball valve mechanism allows the insulfflation and deflation of air from a standard medical syringe. The smaller tube becomes free from the larger tube at 21 cm on the ‘anterior’ surface. The free length of the smaller tube is 20 cm.

22 cm. To inflate the cuff at 12-14 cm, there is another smaller silastic tube attached to the outside of the longer (26 cm) tube. The smaller tube is 2 mm in diameter and is attached to the cuff at one end and a ball valve mechanism at the other end. The ball valve mechanism allows the inflation and deflation of air from a standard medical syringe. The smaller tube becomes free from the larger tube at 22 cm on the ‘anterior’ surface. The free length of the smaller tube is 20 cm.

23 cm. To inflate the cuff at 18-20 cm, there is another smaller silastic tube attached to the outside of the longer (26 cm) tube. The smaller tube is 2 mm in diameter and is attached to the cuff at one end and a ball valve mechanism at the other end. The ball valve mechanism allows the inflation and deflation of air from a standard medical syringe. The smaller tube becomes free from the larger tube at 23 cm on the ‘anterior’ surface. The free length of the smaller tube is 20 cm.

23-24 cm. On the ‘anterior’ surface of the larger tube are two additional smaller tubes that enter the lumen of the larger tube. The first, labeled ‘infusion’, is 3 mm in diameter and 10 cm in length. The second, labeled ‘blood draw’ is 1.5 mm diameter and 10 cm in length. The free ends of both of these tubes have the standard medical interlocking system with syringes and intravenous tubing.

24-25 cm. Three holes, 1 cm by 0.5 cm, placed anterior, left posterior and right posterior positions.

Labeling:

The cuffs, as well as their corresponding smaller tubes and ball valve mechanisms, are labeled. The cuff from 1-4 cm is labeled ‘superior’. The cuff from 12-14 cm is labeled ‘middle’. The cuff from 18-20 cm is labeled ‘inferior’.

The larger, 26 cm, tube is labeled; ‘superior’ at the 5 cm and ‘inferior’ at 24 cm. From 15-17 cm the larger tube is labeled; ‘anterior’, ‘posterior’, ‘left’ and ‘right’ corresponding to usual surgical practice.

How it is Made:

‘P.A. Link Off’ Vascular Clamp

A right-angled vascular clamp. The length of the clamp is 5 cm with a 1 cm superior angled lip. Between the each limb of the clamp is space for a 3 mm tube, 1.5 mm tube, and three 2 mm tubes.

How it is Used:

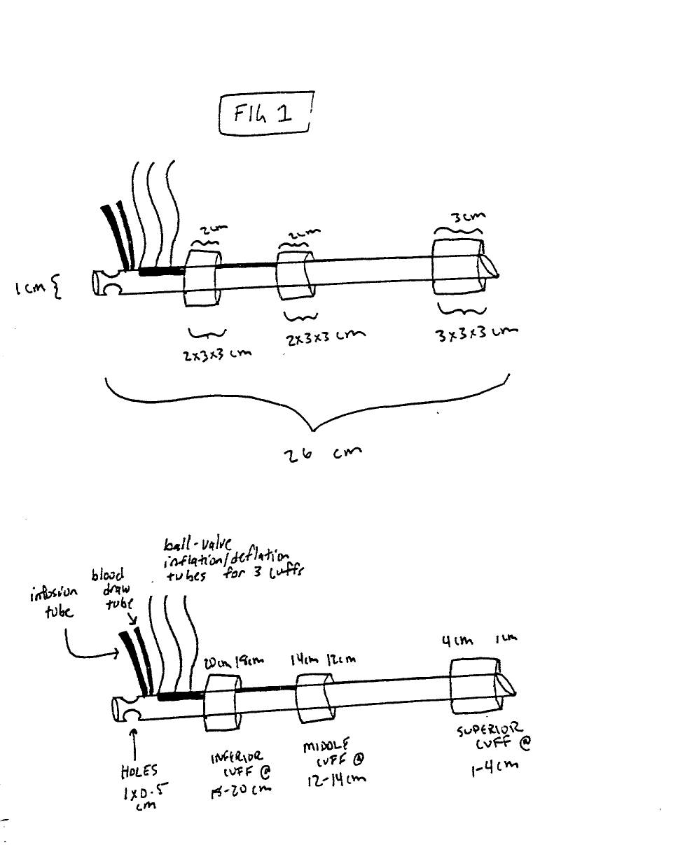

The P.A. Link Off shunt as shown in FIG. 1.

In instances where bleeding cannot be controlled from the inferior vena cava or the post hepatic vena cava, whether from trauma or course of disease, the ‘P.A. Link Off’ shunt can be used.

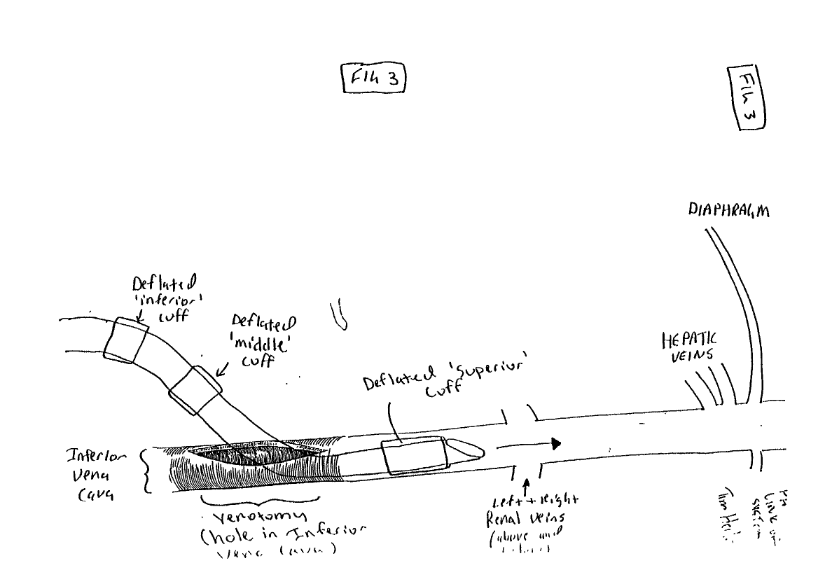

After obtaining distal control of the inferior vena cava, a ‘venotomy’ or a cut is made in the inferior vena cava. The venotomy is inferior to the area of bleeding/hemorrhage. Then the ‘P.A. Link Off’ shunt is placed through the venotomy, into the vena cava, and guided superiorly. (FIG. 3.) Once the ‘P.A. Link Off’ shunt is in place, the ‘superior’ cuff is inflated and gently pulled inferior into the correct position superior to the hepatic veins and inferior to the right atrial opening.

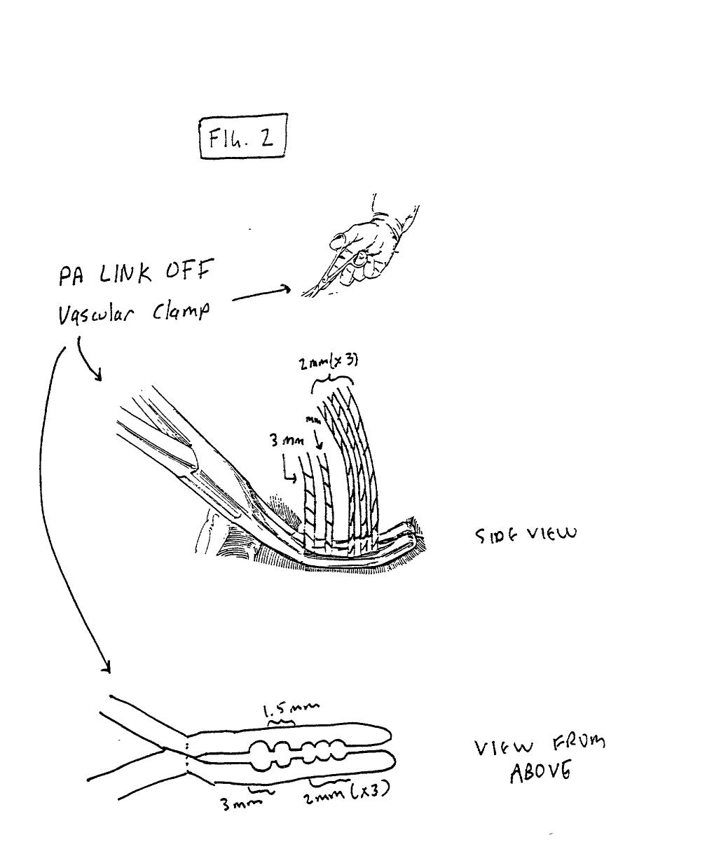

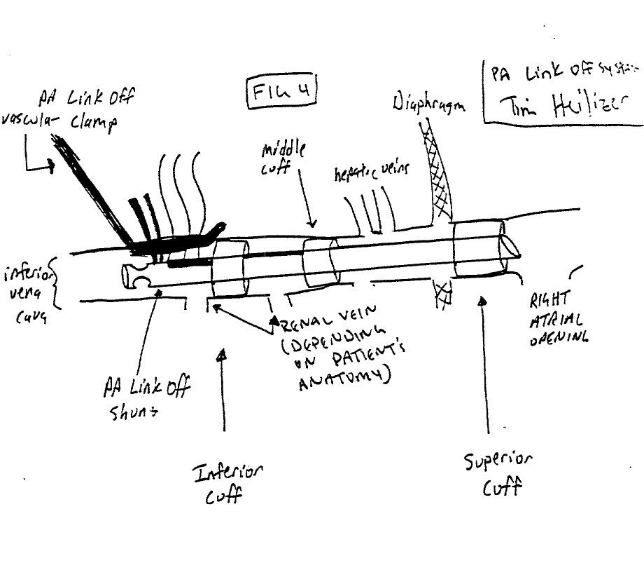

Then the ‘middle’ or the ‘inferior’ cuff is inflated directly superior to the renal veins. Which cuff is inflated depends on the patient's anatomy. (FIG. 4.) The three cuff tubes, ‘infusion’ tube and ‘blood draw’ tube are brought out through the venotomy and the ‘P.A. Link Off’ vascular clamp. The clamp is then closed. (FIG. 2.)

Once the ‘P.A. Link Off’ System is in place, blood flow is diverted from the injured vena cava or post hepatic vena cava, through the ‘P.A. Link Off’ shunt. This allows the surgeon time to fix the injury. The ‘infusion’ tube may be used for intraoperative high volume resuscitation and the ‘blood draw’ tube may be used for intraoperative blood draws.

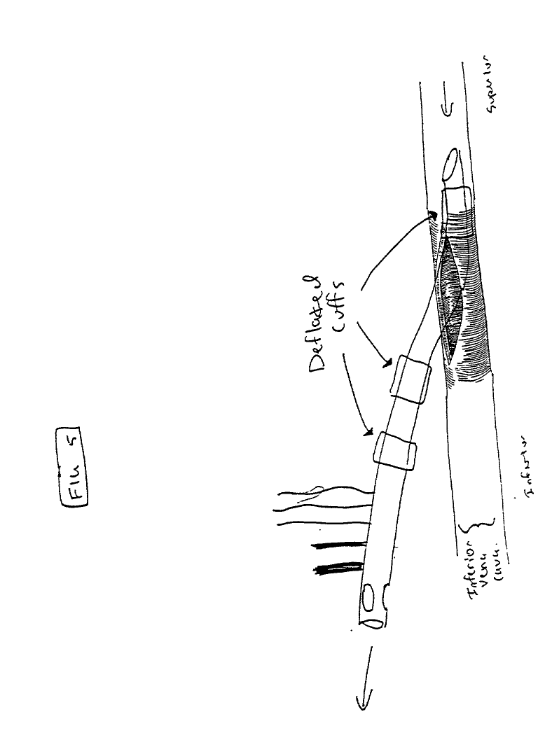

At the completion of the repair, the ‘P.A. Link Off’ system is removed in the reverse fashion from its application. (FIG. 5.)

After distal control of the inferior vena cava is achieved again, the vascular clamp is removed. The inflated cuffs are completely deflated. The ‘P.A. Link Off’ shunt is removed through the venotomy.

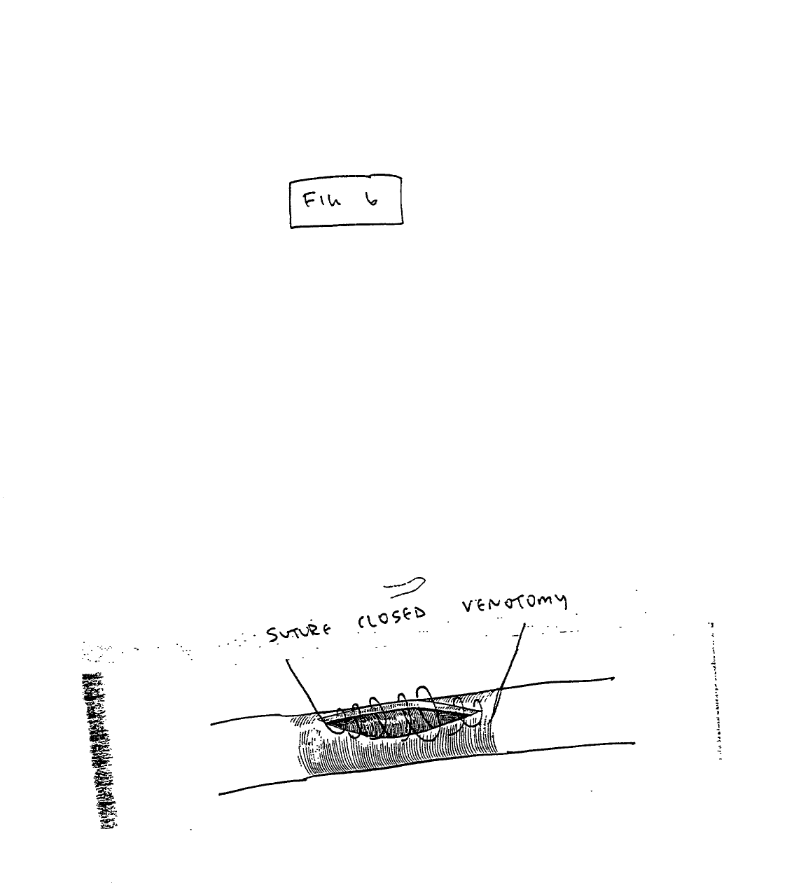

Then the venotomy is repaired. (FIG. 6.)

1. The subject matter, which I regard as the invention, are: The large silastic shunt with multiple cuffs, allowing the isolation of the inferior vena cava and/or the post hepatic vena cava. In addition, the vascular clamp which allows control of the vena cava without compromising the lumens of the smaller tubes from the shunt.
